(19) United States
(12) Patent Application Publication
(10) Pub. No.: US 2012/0101407 A1
(43) Pub. Date: Apr. 26, 2012

(54) APPARATUS AND METHOD FOR PREPARATION OF SMALL VOLUME OF SAMPLES

(75) Inventor: Anthony P. Chan, Tahoe City, CA (US)

(73) Assignee: INOVX, LLC, Tahoe City, CA (US)

(21) Appl. No.: 13/279,149

(22) Filed: Oct. 21, 2011

Related U.S. Application Data
(60) Provisional application No. 61/405,596, filed on Oct. 21, 2010.

(51) Int. Cl. A61B 10/02

(52) U.S. Cl. 600/576

(57) ABSTRACT

A method and device for sample preparation. A capillary having adherent internal desiccated reagent is held by a housing, with one capillary end extending from the housing. A dock on the housing allows a dispense module to be docked onto the housing and dispense the contents of the capillary.

### CROSS-REFERENCE TO RELATED APPLICATION

This application claims priority from provisional application Ser. No. 61/405,596, filed Oct. 21, 2010.

### TECHNICAL FIELD

This invention relates to the collection and subsequent preparation low volume samples for optical assay, such as biological samples.

### BACKGROUND OF THE INVENTION

Accurate and rapid counting of cells or cellular moieties in biological fluids is a necessity in the biomedical, pharmaceutical, environmental and other fields. It is becoming increasingly important to be able to perform rapid on site quantitative testing. For example, in Africa the majority of AIDS infected patients are in remote villages which can be 8 to 10 hours on foot from test clinics in major cities. Consequently, patients do not get diagnosed and do not get treatment for their disease. It is important to be able to test these patients rapidly, at low cost at their villages. On site testing where there are limited lab resources is a challenge; everything needed for the test must be provided on site. For AIDS diagnosis an absolute CD4 count is required and treatment begins when the cell count is <200 cells/uL.

If only a few uL are available for assay, accurate enumeration of cells is difficult for many 5 analytical systems.

U.S. Pat. No. 6,852,527 discloses an apparatus and method to accurately measure the number of cells in biological fluids with a low or very low cell concentration or with low volumes.

The general requirement for assays includes the following steps:

1. Withdraw sample from a sample source.
2. Dispense the sample into a reaction container.
3. Dispense some combination of reagents into the assay container, including some type of detection agent.
4. If required, concentrate or dilute the sample.
5. Assay the reaction mixture and count resulting detected targets (e.g., labeled cells.)

The drawbacks of such a system include:
potential exposure during various steps to pathogenic agents (e.g., virus, bacteria, toxins, etc.)
potential contamination of the sample during each transfer.
relatively large requirements of reagents and samples, and considerable waste of sample.
time and effort required for each step is not efficient.
time required for sample preparation risks alteration of the sample (by bacterial proliferation, changes to unstabilized cell surface antigens, etc.)
requires trained technicians for blood draw
requires lab facility such as supplies (reaction tubes, reagents) and instruments (pipettes, pipette tips, etc.)
difficult to perform at remote locations with limited resources.

There exists a need for a simplified device and system for the preparation of samples for on-site testing where lab resources are not available.

### SUMMARY

The disclosed embodiments include a sample preparation device including a cartridge housing having a capillary tube held within the cartridge housing and having an open capillary tube end extending from one end of said cartridge housing. The capillary tube has adherent reagent at a selected concentration for the capillary volume of the capillary tube. The embodiments also utilize a resilient dispenser dockable onto the cartridge housing for dispensing the capillary tube. This resilient dispenser may, for example, be a separate dispense module that a user may bring into contact with the housing. Alternatively, the resilient dispenser may be a dispense bulb mounted on the housing. The housing also includes an integrated dock shaped and positioned to allow the resilient dispenser to dock with the housing and dispense a fluid from said capillary. This dock may be shaped and proportioned like the upper end of a pipettor tip, to allow use of standard pipettors in dispensing liquid from the housing. Alternatively, the integrated dock may be detents on a housing section or plunger mounted to the housing section.

In some embodiments the resilient dispenser includes an internal membrane and a liquid within the resilient dispenser by the internal membrane. Such an internal membrane is able to be ruptured by pressing on the resilient dispenser such that liquid flow from a resilient dispenser opening.

In other embodiments the housing also includes a lance held on the housing such that a lance tip extends from the housing. Such a lance may be retractable. The device may also include a hinged safety flap movable from a first position in which said safety flap covers the lance tip and the capillary tube open end and a second position in which the lance tip and the capillary end are exposed. The housing also may include a means for retaining the safety flap.

In another embodiment the resilient dispenser includes a plunger having an apical bulb. Lowering an upper section of the plunger into a lower section of the housing (such as a retaining barrel of the housing) allows fluid communication through an upper open end of the capillary tube. The bulb may then be deformed, as by squeezing the bulb, to displace the contents in the capillary tube if said plunger has been lowered. If such a bulb contains a liquid, then the liquid will act to displace the contents of the capillary tube.

### BRIEF DESCRIPTION OF THE DRAWINGS

FIG. 1 is a front perspective view of an embodiment of a cartridge and a wash module embodiment.

FIG. 2 is a cross section of the devices of FIG. 1.

FIG. 3A is a view of the device of FIG. 1 15 with the safety flap open.

FIG. 3B is a view of the backside of an embodiment of a collection cartridge.

FIG. 4 is a front perspective view of an embodiment of a cartridge dispensing into a test cartridge.

FIG. 5 is a front, perspective view of an alternative embodiment of a sample preparation device.

FIG. 6 is a cross section of the device of FIG. 5.

FIG. 7 is a front perspective view of the capillary barrel.

FIG. 8A is a front view of the sample preparation embodiment shown in FIG. 5 used in a test tube to capture a sample.

FIG. 8B is the sample preparation device shown in FIG. 8A with the plunger collapsed.

FIG. 8C is the sample preparation device shown in FIG. 8B with the bulb compressed and a prepared sample being dispersed into a test cartridge.

### DETAILED DESCRIPTION

The disclosed embodiments provide a simple low cost means to collect samples combined with reagents and dispense into an optical analytical system. The disclosed embodiments enable rapid quantitative testing to be done at patient side, in the field and where there are no lab facilities.

### Inovx Sample Preparation Cartridge

To effectively perform patient sample testing as well as other testing, all sample preparation steps, such as patient sample collection and sample preparation with assay reagents, must be done on site. Conventional sample preparation method using pipettes, reagents and other lab supplies to prepare patient sample prior to analysis is cumbersome and wasteful and requires a skilled technician. Present embodiments provide a pre-analysis cartridge that integrates all necessary functions and reagents required for sample preparation in a compact disposable cartridge to make patient side testing simple and effective.

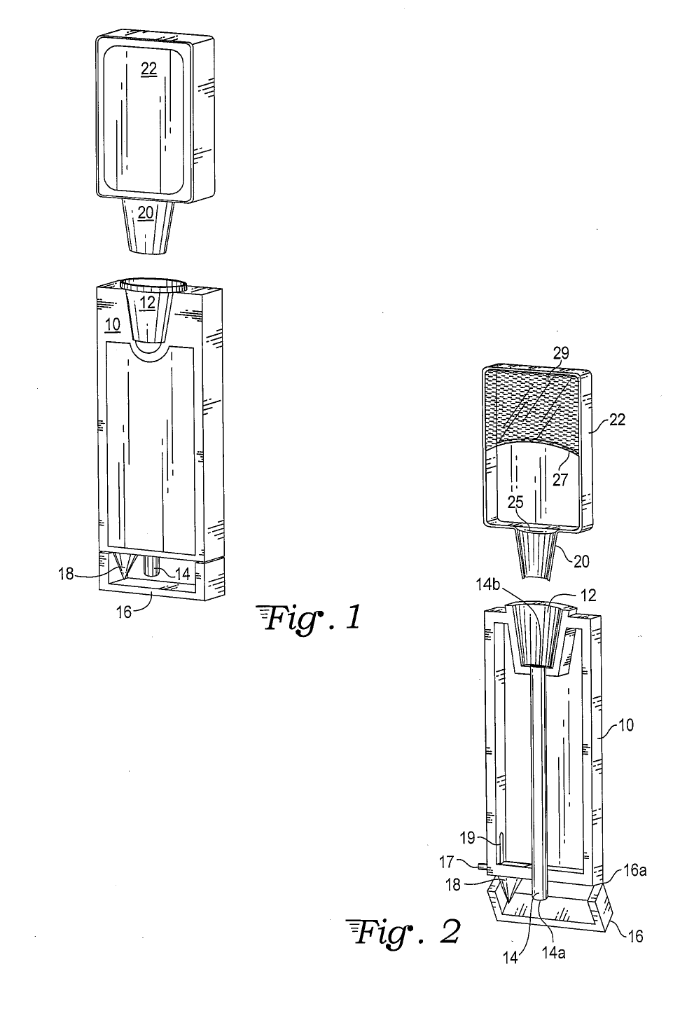

One embodiment is shown in FIG. 1. In this embodiment there are no valves and the cartridge has a simple construction. A safety flap 16 protects the sterility and integrity of a lance 18 and an opening of a capillary dimension tube 14. The lance may not be used in every embodiment, and in some embodiments a lancing element may be incorporated into the end of the capillary tube. The capillary dimension tube 14 may be round or have other shape. The capillary dimension tube 14 has a capillary dimensions such that when the end of the tube is brought into contact with a sample fluid the tube fills by capillary action. Both ends of the tube are open in this embodiment, allowing the tube to fill. This provides a precise amount of sample fluid into the tube contained is within housing 10. At the end of the cartridge opposite the safety flap 16 is a docking port 12. In the illustrated embodiment, the dimensions of this port 12 are similar to the dimensions of a pipette tip. This provides a number of advantages including:

1. Possible dispensing using a pipettor tool.
2. Manipulation of the cartridges using robotic tools designed to manipulate pipette tips.
3. Use of port 12 as a mixing chamber. A pipettor device with an attached tip would dispense a dilution or wash solution into the port. The pipettor would eject the tip, seal onto the port, and then could be used to mix the sample and wash solution. The pipettor then may dispense the solution from the opposite end of the capillary dimension tube.

In this manner the sample collection cartridge would function as a specialized pipette tip. Like pipette tips, these devices could be distributed in sealed boxes, and fabricated from plastic material allowing the device to be sterilized (as by using an autoclave for steam/pressure sterilization or other means).

This device could also have a wash reagent module 22 having a dispense tip 20 which fits into docking port 12. The capillary dimension tube 14 in this embodiment terminates at the bottom of docking port 12, as is shown in FIG. 2. The mixing of the sample and the reagent (such as a fixative, buffer, diluent, lysis agent, or other reagent). The liquid within the reagent module 22 could simply be dispensed by squeezing this module, which may be made of a resilient material. Mixing of the liquid within the module could take place within the module, within a docking port 12, or even within the cartridge or other labware onto which the sample/reagent mix is dispensed.

In some embodiments the capillary dimension tube 14 is coated with a dried reagent for performing a desired assay. For example, the capillary dimension tube 14 could be coated with EDTA, an anti-CD4 antibody/Alexa 610 conjugage (PE, Foster City, Calif.) and an anti CD14 antibody/Cy 5.5 conjugate (PE, Foster City, Calif.). Once the sample is introduced into the tube, the dried reagent goes into solution. Incubation may take place within the tube in about two minutes. The sample may then be dispensed directly into the analysis cartridge, slide, injection post, or other labware or instrument onto which the sample is dispensed.

With reference to FIG. 2, the cross section of the sample preparation device shows that mounted shows that mounted within housing 10 is capillary dimension tube 14. Capillary dimension tube 14 has a first open end 14A and a second open end 14B. Flanking capillary dimension tube 14 is lance 18. Lance 18 may be used to prick a patient (for example a patient's finger. And the resulting small amount of blood would be drawn into capillary dimension tube 14 by capillary action. The volume of capillary dimension tube would be known and the volume of patient sample collected would also be known. Coating the inside of capillary dimension tube 14 in some embodiments may be desiccated assay reagents. The collected patient's sample cause these reagents to go into solution and the targeted cells or other sample constituents would then be labeled by the sample reagents.

The lance 18 is shown mounted on a lance retraction slide 17. Lance retraction slide 17 may move along lance retraction guide 19. This allows lance 18 to be fully retracted into the interior of housing 10.

The integrity of the lance 18 and capillary dimension tube 14 is protected by safety flap 16. Safety flap 16 is attached to housing 10 by hinge 16A. As shown in FIG. 2, safety flap 16 may be retracted back on hinge 16A exposing lance 18 and capillary dimension tube 14. At the second end 14B of capillary dimension tube is docking port 12. This allows docking with dispense tip 20 of wash reagent module 22. The interior of wash reagent module 22 may include a membrane 25 covering the upper end of dispense tip 20. In some embodiments wash reagent module 22 will be filled wash fluid/diluents 29. A mid-line membrane 27 may keep this liquid confined to an upper area within wash reagent module 22. By simply squeezing wash reagent module 22 the user would cause mid-line membrane 27 to rupture and the wash fluid/diluents 29 to flow dispense tip 20.

The drawing of FIG. 2 should make apparent that various alternatives are possible. If wash reagent module is empty, the docking of dispense tip 20 into docking port 12 would allow the contents of capillary dimension tube 14 to be dispensed simply by squeezing the resilient sides of wash reagent module 22. In a second alternative, if the lower end of wash reagent module is empty and dispense tip 20 is docked with docking port 12 this lower portion of wash reagent module may be squeezed prior to docking and then upon docking the vacuum created will suck the sample from capillary dimension tube 14 into the volume of dispense tip 20. Membrane 25 may be a rupturable membrane or may be a membrane having a slit to allow air and liquid to pass through while maintaining device sterility. The patient sample within dispense tip 20 may be then be combined with a known volume of diluent by rupture of mid-line membrane 27 causing wash fluid/diluents 29 to flow into dispense tip 20.

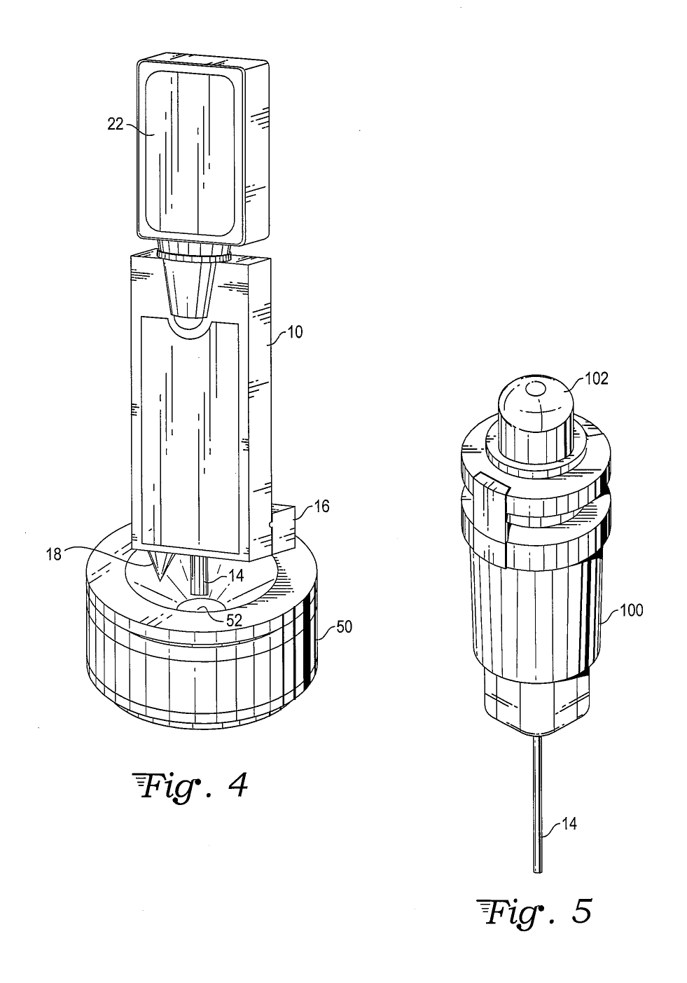

In another alternative the wash reagent module 22 is entirely filled with a diluent. Upon docking of dispense tip 20 with docking port 12 and squeezing the resilient sides of wash reagent module 22 the fluid from wash reagent module 22 would wash the sample and sample reagents into a receiving reservoir of the sample assay device as shown in FIG. 4. Intubation may take place within the capillary tube, with the dispense tip, or within the assay device.

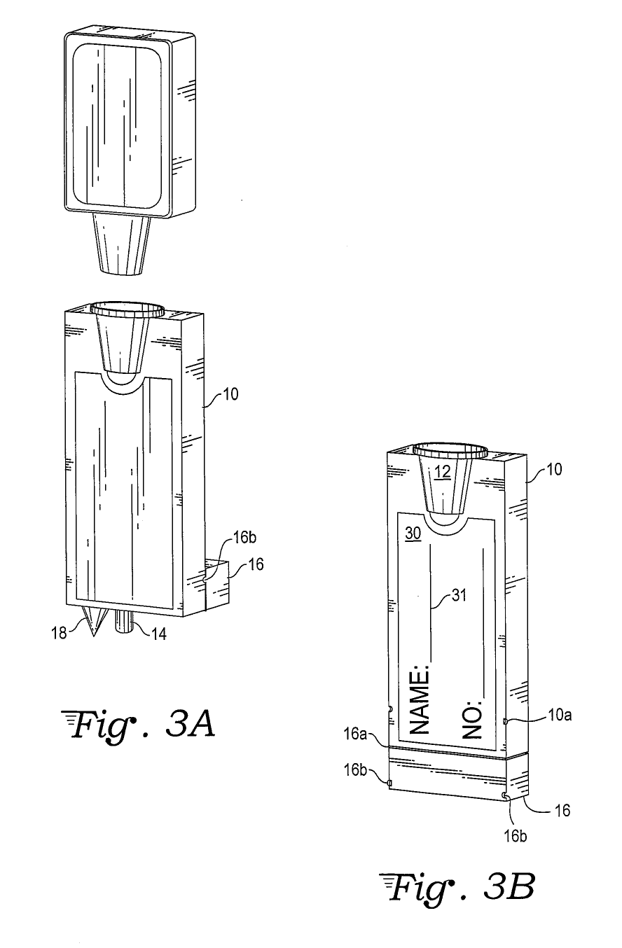

With reference to FIG. 3A, the capillary dimension tube 14 and lance 18 are shown exposed. The safety flap 16 is shown having a protrusion 16B that fits into a receiving dent in housing 10. As shown in FIG. 3B, the receiving dent 10A receives protrusions 16B when safety flap 16 is rotating on hinge 16A. Hinge 16A may be a simple location of a reduced thickness in the resilient material of which housing 10 is manufactured. This would allow housing 10 to be made of injection molding. Capillary dimension tube 14 and lance 18 will subsequently be added to this injection molding piece. This makes the device rather simple and low cost.

Again with reference to FIG. 3B, the backside of the housing 10 shows a label 30 containing written information 31. This information may include barcode or other identifying device.

With reference to FIG. 4, a sample preparation device is shown in which the wash reagent module 22 is mounted onto the housing. The safety flap 16 is retracted such that the end of the capillary dimension tube 14 is able to be brought onto a cartridge 50. Cartridge 50 has a well 52 into which the prepared sample may be deposited by squeezing the resilient walls of wash reagent module 22. Lance 18 is shown extended but may be retracted when dispensing.

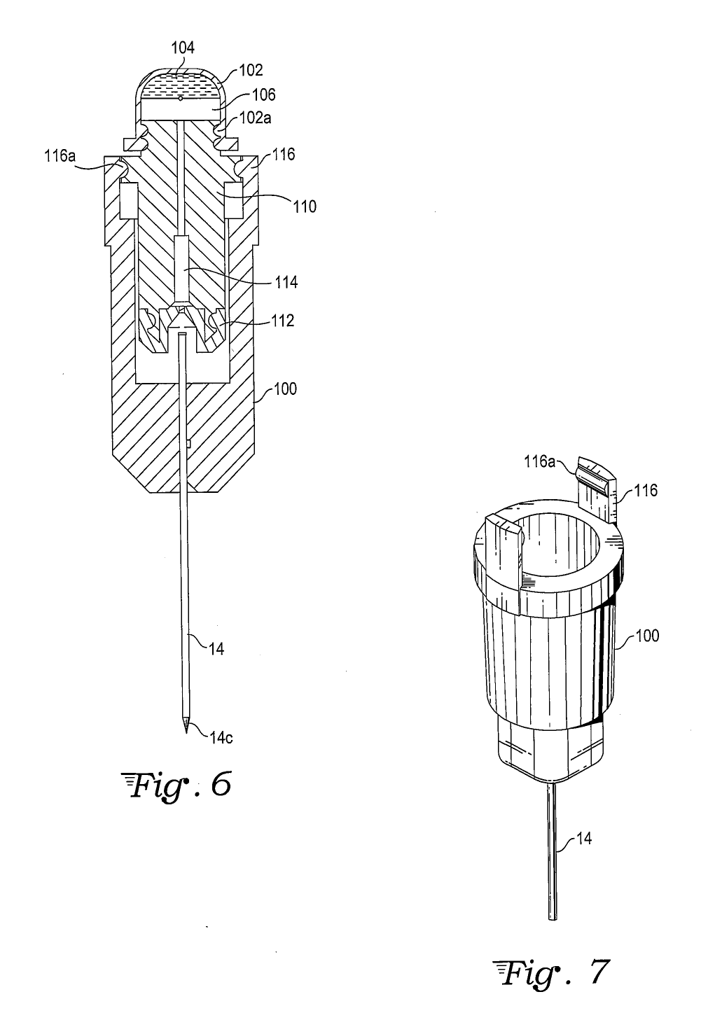

An alternative embodiment is shown in FIG. 5, this embodiment includes a barrel 100, a top bulb 102, and a capillary dimension tube 14. The workings of elements of this embodiment are shown in FIG. 6.

With reference to FIG. 6, the sample preparation device embodiment of FIG. 5 shown in cross section has a capillary dimension tube 14 mounting in barrel 100. The capillary dimension tube 14 may be affixed by adhesive, tightly press-fit in, or mounted by other means. As shown in FIG. 7 the barrel 100 having capillary 14 includes a pair of arms 116, each of which has a horizontal lip 116A. Returning to FIG. 6, these arms 116 are sufficiently flexible such that a user can depress a plunger 110. This causes the seal 112 which is mounted to the bottom of plunger 110 to move downward toward the open upper end 14B of capillary dimension tube 14. Capillary dimension tube 14 would then pierce seal 112 and extend into chamber 114. A user may then squeeze bulb 102 releasing reagent 104 into air space 106 down tube 107. The reagent 104 would then displace the sample from capillary dimension tube 14. In this embodiment capillary dimension tube 14 is shown having a lance tip 14C to allow the capillary tube to also be used to prick a patient to obtain a sample.

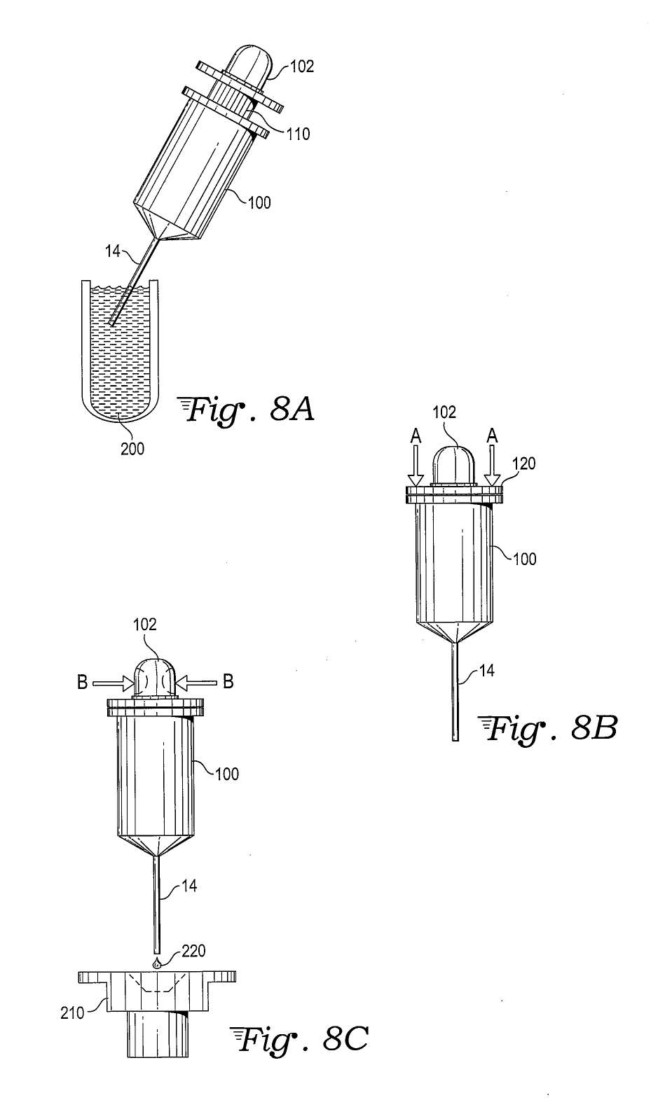

FIGS. 8A-8C illustrate the use of the embodiments of FIGS. 5 and 6 in processing a sample. With respect to FIG. 8A, a test tube 200 holds a sample a liquid to be tested. When capillary tube 14 is introduced into the liquid in tube 200, the liquid is drawn by capillary action to fill the volume of capillary tube 14. Capillary tube 14 is held on held on barrel 100 onto which is mounted plunger 110 and bulb 102.

With respect to FIG. 8B, this shows the plunger being depressed by pressing down upon annular platform 120 to collapse plunger 110 of FIG. 8A into barrel of 100. This is done simply by pushing the annular platform 120 in the direction shown by arrows A. During this step bulb 102 is not depressed.

To dispense the sample the bulb 102 is pressed as shown in FIG. 8C indicated by arrows B. This drives the reagent within bulb 102 into a internal passage way within barrel 100 displacing the volume of capillary tube 14 as sample drop 220 into test cartridge 210.

### White Cell Preparation Cartridge

There are two sections to the cartridge assembly, the body which holds the capillary for sample collection and the plunger in which the reagent is encapsulated.

For the white cell count preparation cartridge, a 5 uL capillary is used for sample collection. 20 uL of Popidium Iodide (PI) is loaded into the reagent chamber (as seen in FIG. 6).

Using the capillary a precise amount of sample is drawn. Then the barrel and plunger are collapsed together. This action inserts the capillary into the reagent chamber. The bulb is then compressed to dispense the reagent and the sample on to the Inovx Test Cartridge. The PI stains the nucleus of the white cells collected on the surface of the test cartridge. The test cartridge is then loaded into the optical analysis unit for analysis.

### Red Cell Preparation Cartridge

For the Red Cell count preparation cartridge, a 5 uL capillary is used for sample collection. 20 uL of PBS is loaded into the reagent chamber, as illustrated in FIGS. 8A-8C.

There are two options for Red cell testing:
1. Pipette 10 uL of cerebral sample spinal fluid into a tube. Add 10 uL of Glycophorin/PE-Cy5. Incubate for about 3 minutes. Draw incubated sample into the cartridge capillary. Wait 2 minutes. Using the bulb, dispense PBS wash and sample onto the Test cartridge for testing in the analyzer.
2. In this option, the reagent is initially dried in the cartridge capillary. 20 uL of PBS is loaded into the reagent chamber. Draw CSF sample into the capillary. The sample mixes with the pre-dried reagent as it enters the capillary. Incubate for 2-3 minutes. Collapse the body and plunger together. Dispense the PBS and incubated sample on to the Test cartridge for analysis in the analysis system.

The use of the collection cartridge requires a simple manual step to complete the assay.

Typical pre-analysis steps needed for quantitive analysis are:

1. Draw patient blood sample
2. Pipette desired volume of sample
3. Pipette reagents to sample
4. Incubate for a length of time (10 mins.)
5. Additional reagent steps, (wash, lyse, etc.)
6. Load sample onto a slide or other labware and place in an analyzer for analysis. (Alternately, the cell sample may be injected into an analytical instrument, (e.g., flow cytometer)).

The method using the disclosed embodiment:

1. The disclosed cartridge provides a retractable lancet for finger stick to obtain patient blood sample
2. It includes a micro channel capillary to collect precise amount of sample. The capillary fills by capillary action and stops filling at the end of the length. Thus, the length of the capillary defines the volume of sample captured, without need for any additional mechanism to drive sample flow.
3. The reagents needed for the assay are dried within the capillary and mixes with the blood as it fills the capillary.
4. Incubation time is greatly reduced (2 mins.) from (10 mins.) for conventional method (in a test tube). The need for pipetting or other processes is also reduced.
5. An additional reagent pouch is provided for lyse, wash or other solutions/diluents if needed.
6. As shown in the embodiment of FIG. 4, the reagent module washes the incubated sample on to the test cartridge for analysis in an analytical device.
7. The single use/single assay cartridge can be safely disposed. Minimization of the cartridge material requirements makes risks of contamination lower, and reduces the burden of proper disposal.

The cartridge can be configured for a variety of specific assays. Any reagent that is dryable onto the surface of the tube is adaptable, as is any detectable label. These could include optical detection chemestives including fluorescent, colorimetric, luminescent assays, or radiological and other labels. Capillary volume can be tailored for each test. It could also be possible to use multiple capillary tubes in a single cartridge if additional volume of sample is needed. The cartridge could also include multiple capillaries for multiple assays on the patient sample. Thus a single lance would provide enough blood for several tests.

Each test uses a minimum amount of sample compared to a typical blood draw. Minimized sample requires minimum amount of reagent. This lowers cost per test. The cartridge is made of inexpensive materials (the housing is plastic) and be made at low cost. Self-contained single use dry reagent cartridge enables convenient distribution and storage, and could be ideal for use in remote locations where storage of reagents in a controlled environment is not available. No refrigeration is required. This design reduces the risks of bio-hazards.

The disclosed system allows development of a variety of tests using the same modular components. Wash modules and capillary tube unit could be sold separately, wash modules may be used for multiple assays. Other known cartridges in current use commonly use the analyzer to actuate valves and piercing mechanisms to obtain a precise amount of sample, dispense reagents and transport sample to analysis location. This approach makes the cartridge complicated, expensive and unreliable and not easily adaptable for other tests. It also makes the analyzer more complicated and expensive. In contrast the disclosed embodiments are inexpensive components and do not require the instrument to provide any sample prep functions.

Obtaining blood sample from a finger stick eliminates the need for a trained nurse to be present for a puncture blood draw.

Using a minimum volume of blood sample, i.e., 1 uL has added advantages since it reduces the amount of reagents needed for the assay consequently reduces the cost per test. Small sample size also presents a detection challenge particularly for samples with low concentration of cells.

The cartridge may be dispensed into an assay device, such as the once disclosed in U.S. Pat. No. 6,852,527, hereby incorporated for all purposes herein. The invention could be characterized in a number of ways, including:

1. A device for obtaining a sample. A capillary is used to capture a precise amount of sample, and within the capillary a dried reagent is included in an amount sufficient to identify targets within the sample.
2. The device above, also including a reagent module configured to dock with the collection cartridge, and also configured to allow manual dispensing capillary by the reagent module. The reagent module could be the illustrated squeeze bulb, a syringe, or other device.
3. A method to capture a desired volume of sample and incubate this sample with reagents that label targets within the sample.
4. The method above, further including dispensing the sample into an analytical device that measures all targets within a sample. This device could be the assay device of U.S. Pat. No. 6,852,527. This device allows all the targets to be visualized at one optically level surface, providing all targets in a field of view. Further, the device has, a well over the sample collection membrane. Incubation could take place within this well. The suction requires to move the targets onto the membrane could be simple centrifugal rotation or vacuum force. A simple suction bulb should be able to draw sample onto this membrane. The assay measures the number of targets in a precise volume. The membrane could be system.

What is claimed is:

1. A sample preparation device comprising:
a cartridge housing;
a capillary tube held within said cartridge housing and having an open end extending from one end of said cartridge housing, said capillary tube having an adherent reagent at a selected concentration for a capillary volume of said capillary tube;
a resilient dispenser dockable onto the cartridge housing for dispensing said capillary tube; and
an integrated dock on said cartridge housing, said dock shaped and positioned to allow the resilient dispenser to dock with said housing and dispense a fluid from said capillary.

2. The device of claim 1, wherein said resilient dispenser includes an internal membrane and a liquid within the resilient dispenser and retained by said internal membrane, wherein said internal membrane is able to be ruptured by pressing on the resilient dispenser such that liquid flow from a resilient dispenser opening.

3. The device of claim 1, wherein said integrated dock is shaped and proportioned to confirm to a shape and proportion of a standard pipette tip.

4. The device of claim 1, further including a lance held on said housing such that a lance tip extends from said housing.

5. The device of claim 4, wherein said lance is retractable into said housing.

6. The device of claim 4, further including a hinged safety flap movable from a first position in which said safety flap covers said lance tip and said capillary tube open end extending from one end of said cartridge housing and a second position in which said lance tip and said capillary tube open end are exposed.

7. The device of claim 6, wherein said housing includes a means for retaining said safety flap.

8. The device of claim 1, wherein said resilient dispenser includes a plunger having an apical bulb, wherein lowering an upper section of said plunger into a lower section of said plunger allows fluid communication through an upper open end of said capillary tube and wherein deforming said bulb displaces contents in said capillary tube if said plunger has been lowered.

9. The device of claim 8, wherein said bulb includes a volume of liquid and an internal bulb membrane, wherein squeezing said bulb ruptures said membrane.

10. A method for preparing samples comprising:
lancing a patient at a lance location using a housing mounted lance;
drawing a specific volume of blood from said lance location into a housing mounted capillary tube section proximate to said lance;
incubating said specific volume of blood in said tube with a known concentration of adherent desiccated compounds in said capillary tube, said compounds including at least one blood component binding agent and at least one detectable label; and
dispensing liquid from said capillary tube section using a resilient dispenser docked with said housing.

11. The method of claim 10, further including an initial step of retracting a safety flap from said housing to expose a lance tip and an open end of said capillary tube section.

12. The method of claim 10, wherein air is used to dispense liquid from said capillary tube section.

13. The method of claim 10, wherein liquid from within said resilient dispenser docked with said housing is used to dispense liquid from within said capillary tube section.

14. The method of claim 10, wherein dispensing liquid from said capillary tube section using a resilient dispenser docked with said housing includes lowering an upper section of a plunger movable in relation to said housing, into a lower section of said plunger to create a passageway for fluid communication to an upper open end of said capillary tube and deforming a bulb on said housing such that it displaces some internal bulb volume which is communicated to the capillary tube such that contents in said capillary tube are displaced.
